(19) United States
(12) Patent Application Publication
(10) Pub. No.: US 2008/0132783 A1
(43) Pub. Date: Jun. 5, 2008

(54) Pelvis Registration Method and Apparatus

(76) Inventors: Ian Revie, N. Yorkshire (GB); Alan Ashby, York (GB); Mike Slomczykowski, Scholes (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/598,590

(22) PCT Filed: Mar. 7, 2005
(86) PCT No.: PCT/GB05/00841

§ 371 Date: Dec. 13, 2007

Related U.S. Application Data
(60) Provisional application No. 60/575,365, filed on Jun. 1, 2004.

(30) Foreign Application Priority Data
Mar. 5, 2004 (EP) 04251288.9
Mar. 8, 2004 (EP) 04251321.8

(51) Int. Cl. A61B 5/05

(52) U.S. Cl. 600/426; 600/425; 702/19

(57) ABSTRACT

A method and apparatus for registering a pelvis of a subject in a lateral position are described. The method includes the position of at least a first point and a second point in a first plane parallel to a first cardinal plane of the pelvis. Then the position of at least a third point and a fourth point in a second plane parallel to a second cardinal plane of the pelvis are determined, in which the second plane is perpendicular to the first plane. Then the position of a third cardinal plane of the pelvis is determined, in which the third cardinal plane is perpendicular to the first cardinal plane and second cardinal plane. The apparatus includes a computer aided surgery system and an instrument trackable by a tracking part of the system.

The present invention relates to pelvic registration, and in particular to methods, apparatus, computer program code and computer program products for use in registering the position of a pelvis of a subject disposed in a lateral position.

Various methods exist for registering the pelvis of a patient. For example, the position of several points on the surface of the iliac crest and acetabular rim can be determined using a mechanical pointer and then registered with a set of computer tomography (“CT”) images of the pelvis. An intra-operative method can capture fluoroscopic images of the pelvis using a C-arm and then registering the pelvis by matching anatomical features of the pelvis to the CT images including those features. Alternatively, the CT images can be used to determine reference planes of the pelvis. These methods are image based and require the use of pre-operatively, or intra-operatively, captured images.

A non-image based registration method involves determining the positions of a number of points on the pelvis during surgery.

However, there are a number of surgical procedures which required the patient to be disposed in a lateral position rather than a supine position. With the patient in the lateral position, the anatomical points used with the patient in the supine position are not readily accessible by the surgeon. However, not all medical facilities, or operating theatres, have the CT imaging systems required by the above mentioned methods for registering the pelvis in the lateral position. In the absence of a CT imaging system, one approach to registering the pelvis is to register the pelvis with the patient in the supine position and then re-position the patient in the lateral position. However, this approach can be associated with anaesthetic hazards, can lead to problems in maintaining sterility and the position of the pelvis can change, either owing to the handling of the patient or owing to the different loads that the pelvis is under in the supine and lateral positions.

Therefore it would be beneficial to be able to register the pelvis of a patient with the patient oriented a lateral position without the need for imaging equipment.

According to a first aspect of the present invention, there is provided a method for registering a pelvis of a subject in a lateral position. The position of a first point and a second point in a first plane can be determined. The first plane can be parallel to a first cardinal plane of the pelvis. The position of a third point and a fourth point in a second plane can be determined. The second plane can be parallel to a second cardinal plane of the pelvis. The second plane can be perpendicular to the first plane. The position of a third cardinal plane of the pelvis can be determined and the third cardinal plane can be perpendicular to the first cardinal plane and the second cardinal plane.

By determining the position of sufficient points and using geometric constraints the position of a pelvis of a patient in a lateral position can be registered within a frame of reference.

A one of the first and second points or a one of the third and fourth points can be common to the first plane and second plane. In this way the number of points used to register the position of the pelvis can be reduced.

The first cardinal plane can be the pelvic frontal plane. The second cardinal plane can be the pelvic mid sagittal plane. The third cardinal plane can be the transverse pelvic plane.

The position of more than two points in the first or second planes can be determined. The position of a fifth point in the first plane can be determined. The position of a sixth point in the second plane can be determined. The position of at least three points in the first and/or second plane can be determined.

The first plane can be the first cardinal plane. The second plane can be the second cardinal plane.

Determining the position of the first, second, third and fourth points can include tracking the position of an instrument detectable by a tracking system. The instrument can bear a marker detectable by the tracking system. The marker can be wirelessly detectable. The marker can be detectable using electromagnetic radiation. The marker can be detectable using electromagnetic radiation in the radio frequency (RF) part of the spectrum.

The method can include registering the position of the pelvis with a virtual model of a pelvis or an image of the pelvis. This can be of use in computer aided surgery (“CAS”) or image guided surgery (“IGS”) procedures.

The method can further comprise attaching a marker detectable by a tracking system to determine the position of the marker to a specified anatomical feature of the pelvis.

Determining the position of the first, second, third and fourth points can include applying an end of an instrument to respective anatomical features of the pelvis. Determining the position of the first, second, third and fourth points can include subcutaneously applying the end of the instrument. Determining the position of the first, second, third and fourth points includes percutaneously applying the end of the instrument.

The method can further comprise palpating the skin of the subject to identify the anatomical features before applying the end of the instrument to the skin above the anatomical feature.

Determining the position of the third cardinal plane can comprise calculating the position of the third cardinal plane. The position of the third cardinal plane can be calculated using a geometrical constraint only. The geometrical constraint can be that the third cardinal plane is perpendicular to both the first cardinal plane and the second cardinal plane.

The points of the pelvis can be anatomical features of the pelvis or anatomical features which are stationary relative to the pelvis and which are readily accessible with the patient in a lateral position.

The first point can be the spina iliaca anterior superior. The second point can be the middle of the symphysis pubis. The third and/or fourth points can be any points located on the mid-sagittal plane of the pelvis. The third and/or fourth points can be an anatomical feature of a vertebra or vertebrae. The third and fourth points can be spinal bone segments. The third and fourth points can be different sacral and/or lumbar parts of the spine. A one of the third and fourth points can be a spinous process of the L5 lumbar vertebra. Preferably the third and fourth points are spinal processes of the sacral bone. The third point can be the spinous process of the S1 (Sacrum 1) vertebra. The fourth point can be the spinous process of the S2 (Sacrum 2) vertebra. The third and/or fourth points can be selected from the spinal process S1, spinal process S2, spinal process L5, gluteal cleft and any other anatomical point located on the mid-sagittal plane of the pelvis

The point common to the first and second planes can be the symphysis pubis.

A further aspect of the invention provides a system for registering the pelvis of a patient in a lateral position. The system can comprise an instrument for locating a plurality of points on, or adjacent, the pelvis. The position of the instrument can be detectable by a tracking system. The tracking system can be operable to generate a signal indicative of the position of the instrument. A computer system can be provided in communication with the tracking system to receive the signal. The computer system can include a data processing device and a memory storing instructions. The instructions can cause the data processing device to determine the position of a first pelvic plane using a first set of pelvic part positions, determine the position of a second pelvic plane from a second set of pelvic part positions and determine the position of a third pelvic plane. The pelvic planes can be mutually perpendicular.

The instrument can bears a marker detectable by the tracking system. The marker can be wirelessly detectable by the tracking system.

The instrument position signal can comprise at least one data item representative of the position of the instrument. The instrument position signal can further comprise at least one data item identifying the marker.

The tracking system and computer system can be integrated. The tracking system and computer system can be separate.

The first set of pelvic positions can include at least three pelvic positions, or positions adjacent the pelvis, in a first plane parallel to the first pelvic plane, or in the first pelvic plane. The second set of pelvic positions can include at least two pelvic positions, or positions adjacent the pelvis, in a second plane parallel to the second pelvic plane, or in the second pelvic plane. A one of the pelvic positions, or positions adjacent the pelvis, in the first set of pelvic positions and the second set of pelvic positions can be common. The second set of pelvic positions can include at least three pelvic positions, or positions adjacent the pelvis, in a second plane parallel to the second pelvic plane, or in the second pelvic plane. Preferably, the positions adjacent the pelvis are anatomical features which are static relative to the pelvis.

The pelvic positions, or positions adjacent the pelvis, are preferably readily accessible by a surgeon with the patient in the lateral position. That is, the surgeon has access to percutaneously or subcutaneously locate the positions or anatomical features with the patient in the lateral position without moving the patient out of the lateral position.

The first set of pelvic part positions can include the positions of the spina iliaca anterior superior and the symphysis pubis. The second set of pelvic part positions includes the positions of the spinous process of the S1 vertebra and the spinous process of the S2 vertebra. The second set of pelvic part positions can include the positions of the spinal process of any two of the following vertebra: L1, L2, L3, L4, L5, S1, S2 and S3. Preferably the two vertebra are selected from the group comprising: L5; S1; and S2. These latter three points are more readily accessible to the surgeon.

According to a further aspect of the invention, there is provided a method for registering a pelvis of a subject in a lateral position. The position of a first cardinal plane of the pelvis can be calculated or derived using the position of a first point and a second point in a first plane parallel to the first cardinal plane. The position of a second cardinal plane of the pelvis can be calculated or derived using the position of a third point and a fourth point located in a second plane parallel to the second cardinal plane. The position of a third cardinal plane of the pelvis can be calculated or derived. The first, second and third cardinal planes are mutually perpendicular.

Hence, by using geometrical constraints and the positions of points of, or adjacent, the pelvis, the position of the pelvis of a patient in a lateral positions can be derived or obtained.

The first plane can be coincidental with the first cardinal plane and/or the second plane can be coincidental with the second cardinal plane.

The first cardinal plane can be the frontal pelvic plane and/or the second plane can be the sagittal pelvic plane and/or the third plane can be the transverse pelvic plane.

Calculating or deriving the position of the second cardinal plane can also use a one of the first or second points. In this case the one of the first or second points is common to two planes and therefore a reduced number of points are used. The first or second point can be on the symphysis pubis.

According to a further aspect of the invention, there is provided computer program code executable by a data processing device to provide the preceding method aspect of the invention. Also provided is a computer program product comprising a computer readable medium bearing computer program code according to the preceding aspect.

According to a yet further aspect of the invention, there is provided a method for registering the pelvis of a subject in a lateral position. The position of an instrument at a plurality of positions can be detected. The position of a plurality of corresponding anatomical features of, or adjacent, the pelvis can be determined based on the respective detected positions. The position of first, second and third mutually orthogonal cardinal planes of the pelvis can be calculated or derived.

The plurality of positions can comprise at least a first, second, third and fourth position. More than four positions can be detected. The plurality of corresponding anatomical features can comprise a first, second, third and fourth anatomical feature of, or adjacent, the pelvis. The positions of more than four corresponding anatomical features can be determined.

Detecting the position of the instrument can include wirelessly tracking the instrument.

Three of the first, second, third and fourth anatomical features can lie in the same plane.

Calculating the position can further comprise using a one of the first, second, third and fourth anatomical features which does not lie in the same plane and the geometric constraint that a two of the cardinal planes are perpendicular.

An embodiment of the invention will now be described, by way of example only, and with reference to the accompanying drawings, in which:

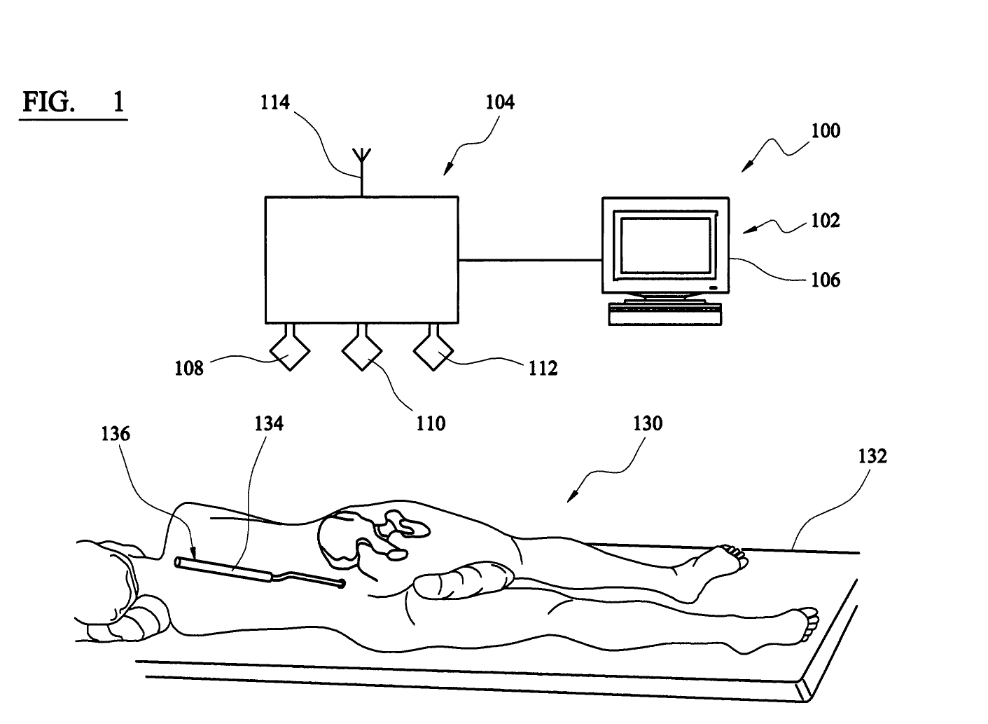

FIG. 1 shows a schematic block diagram of a system for use in registering a pelvis of a patient in a lateral position according to the invention;

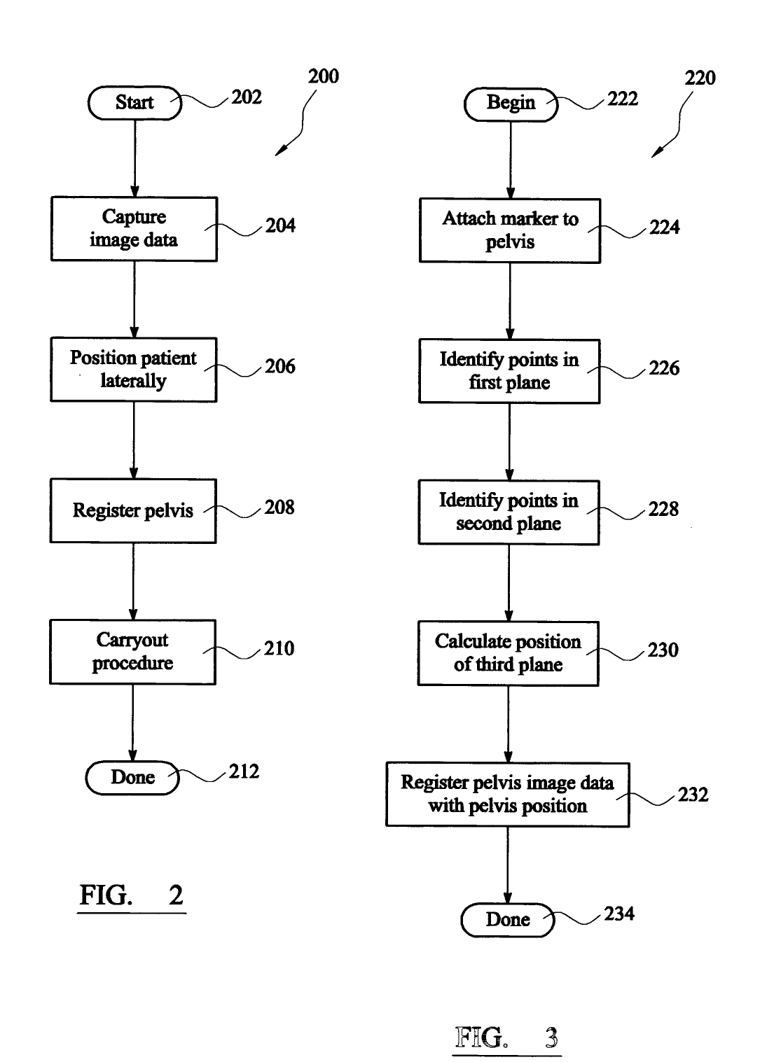

FIG. 2 shows a high level flow chart illustrating a method of carrying out a surgical procedure using the system shown in FIG. 1 and including a method for registering a pelvis of a patient in a lateral positions according to the invention;

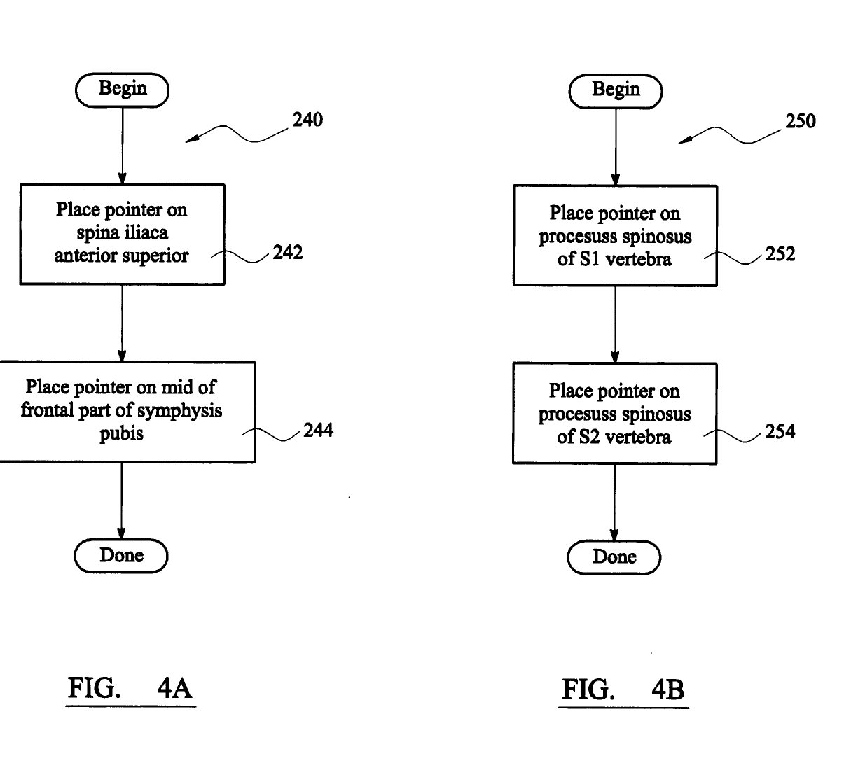

FIG. 3 shows a flow chart illustrating the method for registering a pelvis of a patient in a lateral positions in greater detail;

FIGS. 4A and 4B respectively show flow charts illustrating parts of the method illustrated in FIG. 3 in greater detail;

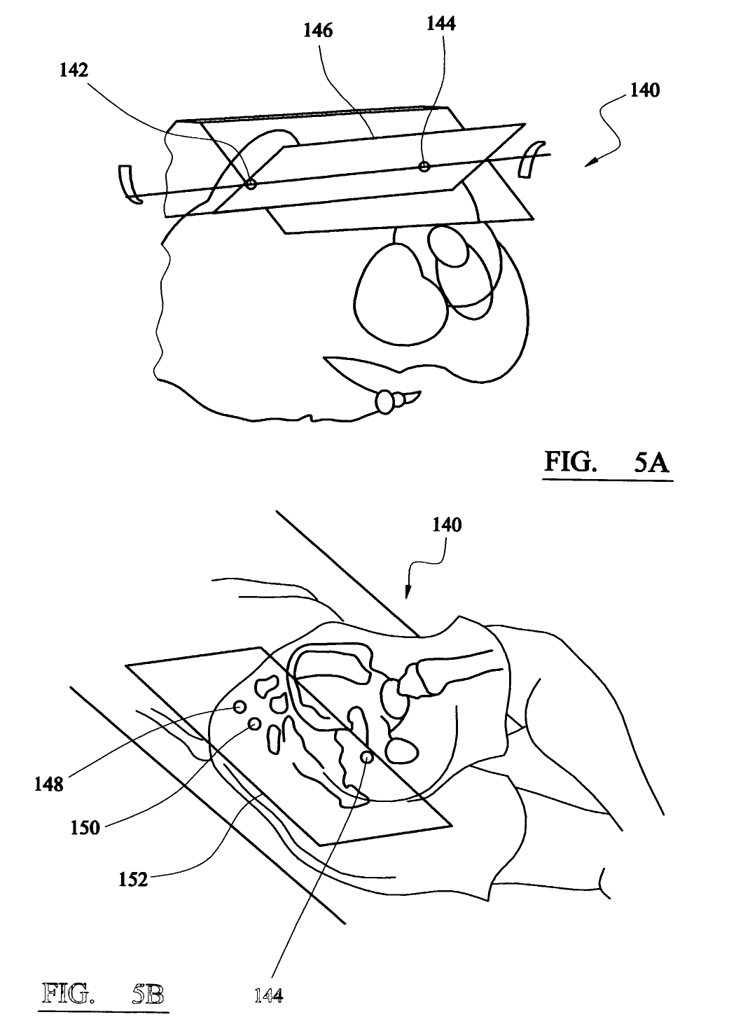

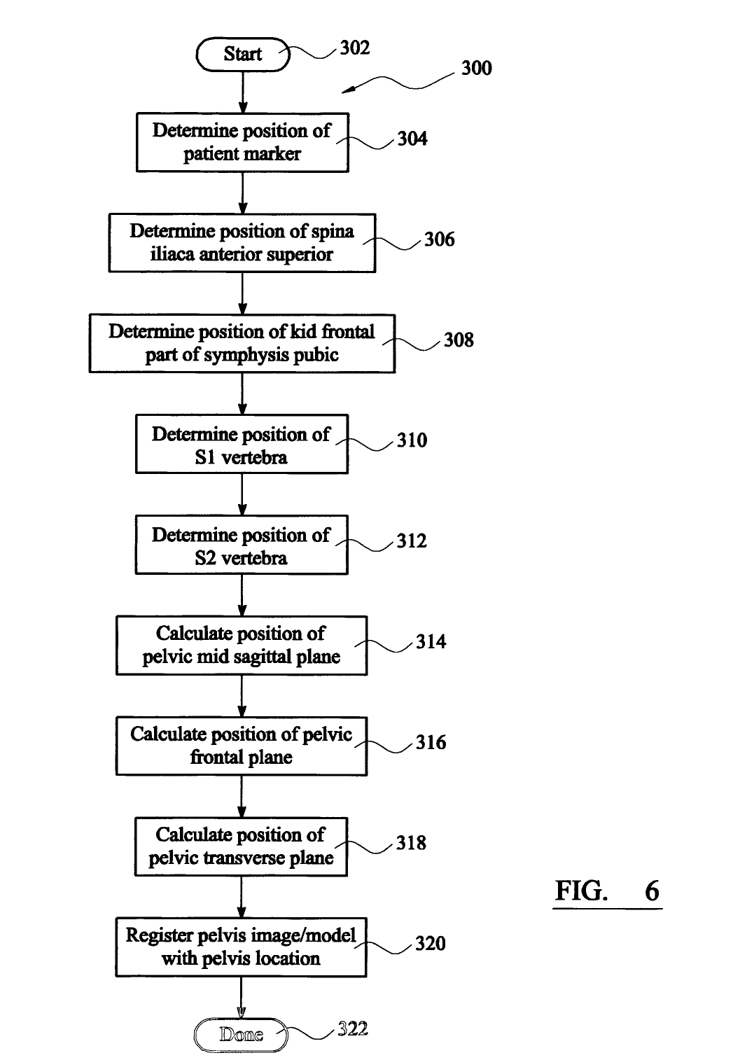

FIGS. 5A and 5B respectively show perspective views of the pelvis of a patient illustrating the location of anatomical points acquired during the method;

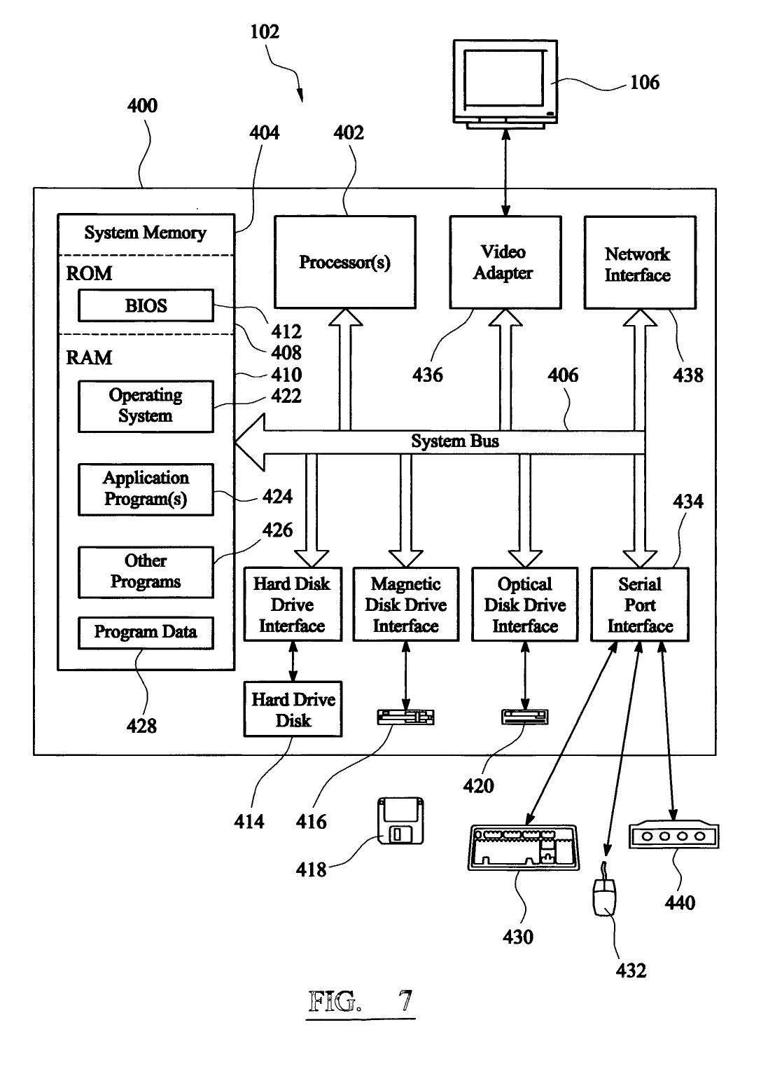

FIG. 6 shows a flow chart illustrating data processing operations carried out by the system shown in FIG. 1; and

FIG. 7 shows a schematic block diagram of a computer part of the system of FIG. 1.

Similar items in different Figures share common reference numerals unless indicated otherwise.

The present invention will now be described in greater detail within the context of carrying out a hip replacement surgical procedure. However, it will be appreciated that the invention is not limited to that particular procedure and can be used in any surgical, or clinical, therapeutic, prophylactic or diagnostic procedure in which it is useful to be able to register the position of the pelvis so as to take advantage of computer aided surgery (“CAS”) and in particular image guided surgery (“IGS”). The invention will also be described within the context of an RF wireless tracking system. However, other tracking technologies and associated marker technologies can be used. For example wire based tracking systems can be used. Also other wireless tracking technologies can be used, such as acoustic based systems, e.g. using ultrasound, and electromagnetic radiation based systems, e.g. using infra-red (IR) and microwave frequencies.

The pelvis can generally be represented as having three mutually perpendicular reference, or cardinal, planes (the transverse plane, the mid-sagittal plane and the frontal plane) which between them define a virtual rectangular space within which the pelvis lies. By determining the locations of these planes, the position of the pelvis can reliably be determined.

With reference to FIG. 1, there is shown a computer aided surgery (CAS) system 100 which includes a suitably programmed general purpose computer system 102 and a tracking system 104. Computer system 102 includes a visual display device 106 and input devices (not shown) by which a user can enter commands to control software being executed by the computer system. The input devices can include a keyboard and pointer device, such as a mouse, and the display device can include a touch sensitive screen.

Tracking system 104 is shown separately to computer system 102 in order to aid clarity of description only and can in practice be integrated into computer system 102. Tracking system 104 uses radio frequency (“RF”) electromagnetic signals to wirelessly communicate with markers including transponders which can be attached to the patient and tools or instruments used by the surgeon. In one embodiment, the tracking system includes three coils 108, 110, 112 which generate an electromagnetic field distribution within a working volume within which the surgical site, i.e. the region around the pelvis, of a patient 130 is located. As illustrated in FIG. 1, the patient or subject is disposed in a lateral decubitus position.

Each marker includes three mutually perpendicular coils which detect the field components at their position within the working volume by induction to generate voltage signals which are processed by a signal processing circuit part of the marker to generate a digital signal. The digital signal is then transmitted by the marker at a different RF frequency and picked up by an antenna 114 of the tracking system. The digital signal is processed by the tracking system to generate signals or data indicating the position and orientation of the tracked marker within the reference frame or co-ordinate system of the tracking system. Each marker also has a unique identifier which is also transmitted to the tracking system to provide a marker ID data item so that the CAS system is aware of which individual marker the position data relates to. Further details of a suitable tracking system and marker are provided in US patent application publication no. US-A-2003/0120150 (U.S. patent application Ser. No. 10/029,473) and international patent application publication no. WO-A1-96/05768 which are incorporated herein by reference in their entirety for all purposes.

As also shown in FIG. 1, a patient 130 is positioned on a table 132 or other support in a lateral decubitus position. The surgeon can use an instrument, in the form of a probe or pointer 134 which bears a trackable marker 136 so that the position of the pointer in the reference system of the tracking system can be determined. In other embodiments, a wireless tracking system can be used, and in further embodiments, the position of the instrument can be determined by mounting the instrument on the end of an articulated arm having transducers at its joints providing signals processable to indicate the position of the arm, from which the position of the instrument can be determined.

The tracking system 104 can provide either signals to the computer system which are processed to provide data items representative of the position of each tracked marker, or alternatively, the tracking system can provide data items directly to the computer system as input to a CAS application. The computer system and CAS application will be described in greater detail below with particular reference to FIGS. 6 and 7.

Firstly, the method of registering the pelvis will be described within the context of a total hip replacement procedure. As indicated above, the method can be used in other procedures also.

With reference to FIG. 2, there is shown a flow chart illustrating a method 200 for carrying out a surgical procedure. The method begins at step 202. Step 204 is an optional step in which image data of the surgical site of the patient can be captured. For example a CT scan of the patient's pelvis can be carried out and the patient body part image data is stored in a suitable format. Other types of patient body part image capture can be carried out at step 204, for example, image data can be captured using X-rays or ultrasound and either digital images can be captured or the captured images can be digitised in order to provided patient body part image data which is stored in a suitable format for subsequent handling, communication and processing.

After pelvis and hip image data has been collected at step 204, at step 206, the patient is provided in a lateral decubitus position in the operating room in which the surgical procedure is to be carried out. It will be appreciated that if the operating room also has imaging apparatus available, then the image capture of step 204 can also be carried out in the operating room. Either way, the image data is collected in step 204 with the patient in the lateral decubitus position. Step 206 therefore merely corresponds to providing the patient in the lateral decubitus position in the operating room.

At step 208, the position of the patient's pelvis is registered with the reference frame, or co-ordinate frame, of the tracking system 104. That is, the CAS system is provided with sufficient information as to the position of various sites on the pelvis, via the tracking system, that the CAS system can determine the position of the pelvis within the reference frame of the tracking system.

The size of the pelvis can also be determined for an image based method in which an image of the pelvis is obtained before registration. For image free methods, the size can also be determined using a morphing algorithm to morph a generic model of the shape of a pelvis into the shape of patient's actual pelvis using the collected points. If morphing is not used, then the method provides the reference planes of the patients's pelvis which can be used to calculate the anteversion and inclination angels for acetabular cup implantation

The step of registering the patient's pelvis with the CAS system will be described in greater detail below. Then, at step 210, the surgical procedure can be carried out using the CAS system to assist the surgeon. The position of the pelvis can be used by surgical workflow, planning and navigation software applications.

For example, a workflow application can be used to guide the surgeon through the steps of a hip replacement operation. The workflow can include steps associated with planning the surgical procedure, such as determining the appropriate positions and orientations for the pelvic and femoral implants using the pelvis position so as to provide appropriate post-operative kinematic performance of the implants. Once the preferred implant positions have been determined, navigation surgical steps can be included in the workflow to assist the surgeon in the correct positioning of tracked tools and instruments. Navigated and image guided actions facilitate the performance of procedural steps associated with inserting and attaching the implants, such as the drilling of pilot and guide holes, the reaming of cavities for receiving implant stems and implant bodies, and the resecting of bone surfaces. It will be appreciated that other navigated and image guides operation steps can be carried out.

Various other navigated and image guided procedures can be carried out during step 210 until the hip replacement procedure has been completed and then ends at step 212. The details of completing the surgical procedure have not been described in detail herein so as not to obscure the nature of the present invention.

FIG. 3 shows a flowchart illustrating a method of registering a pelvis 220 which corresponds generally to step 208 of FIG. 2. The method begins at 222 and at step 224 a marker which is detectable by the tracking system is attached to the patient at the iliac crest to track the position of the pelvis. The marker allows any the intra-operative movements of the pelvis to be compensated for and allows registration to be independent of any patient movement. The pelvis marker can be attached anywhere on the pelvis, however, the iliac crest is preferred. The marker can be attached outside the operating wound, or percutaneously. In one embodiment, the marker is attached to the iliac bone close to the acetabulum and inside the operating wound.

Then at step 226, the surgeon identifies a plurality of points on the pelvis which lie in a first plane which is either parallel to a first cardinal plane of the pelvis or actually lie in the first cardinal plane of the pelvis. The plurality of points are identified by the surgeon placing the tip of a trackable pointer on the pelvis points and the tracking system determining the position of the pointer and therefrom the position of the tip which corresponds to the position of the pelvic points.

FIG. 4A shows a flow chart illustrating a method 240 for identifying points in the first plane in greater detail and which corresponds generally to step 226. The surgeon can identify points percutaneously or subcutaneously. In a percutaneous approach the surgeon palpates the skin in a region over the pelvis in order to find the anatomical point of the pelvis. Once the surgeon has found the point, the surgeon then places the end of the pointer on the skin above the point and tells the computer system to capture that position. In a subcutaneous approach, the surgeon places the pointer directly on the pelvis via a suitable incision or incisions and notifies the computer system to capture the position of that point. A combination of percutaneous and subcutaneous approaches can be used for different points. Points in the second plane also can be identified percutaneously and/or subcutaneously.

At step 242, a first point in the pelvic frontal plane is identified by placing the tip of the pointer percutaneously or subcutaneously on the spina iliaca anterior superior of the patient's pelvis. FIG. 5A shows a perspective view of a part of the patient's pelvis 140 and illustrates the position of the spina iliaca anterior superior anatomical point 142. The surgeon enters an instruction to the computer system, e.g. by touching a ‘button’ displayed on the touch screen 106, to capture the current position of the pointer using the tracking system. Then at step 244, the surgeon places the tip of the pointer percutaneously or subcutaneously on the mid frontal part of the symphysis pubis of the patient's pelvis and the current position of the pointer is captured as described above. FIG. 5A also shows the position of the mid part of the symphysis pubis anatomical point 144. The above two anatomical points of the pelvis both lie in the frontal plane 146. However, without any further information, the position of the frontal plane 146 is not completely defined, and at this stage can be any plane including the two anatomical points 142, 144.

In other embodiments, the anatomical points of the pelvis all lie in a plane which is parallel to the frontal plane and not coincidental with it.

Returning to method 220, at step 228 a plurality of points in a second plane of the pelvis, and which is perpendicular to the first plane, are identified by the surgeon. FIG. 4B shows a flow chart illustrating a method 250 for identifying a plurality of points in the second plane in greater detail. In the described embodiment the second plane is coincidental with, i.e. is the same plane as, a cardinal plane of the pelvis. In other embodiments, the second plane is parallel to the second cardinal plane of the pelvis. The method 250 includes identifying anatomical points that lie in the pelvic mid-sagittal plane.

FIG. 5B shows a perspective view of the pelvis 140 of the patient illustrating a number of anatomical features of, or adjacent, the pelvis. At step 252 the surgeon places the tip of the marked pointer on the procesuss spinosus of the S1 vertebra 148 of the patient. In another embodiment the spinous process of the L5 vertebra is used instead. Either a percutaneous or a subcutaneous approach can be used as described above. The computer system then captures the identified position and determines the position of the point as described above. Then at step 254 the surgeon places the tip of the marked pointer on the procesuss spinosus of the S2 vertebra 150 of the patient and the position of the point is captured and determined as described above. Using anatomical features of the S1 and S2 vertebra is not necessary but is advantageous as their position is fixed relative to the pelvis. Anatomical points 148 and 150 both lie in the mid sagittal pelvic plane 152, and point 144 also lies in this plane.

In other embodiments, other anatomical features that are accessible with a patient in a lateral position can be used. In general the positions of at least two points from the mid sagittal plane, or parallel to the mid sagittal plane, are captured.

Returning to FIG. 3, the pelvic registration method 220 proceeds by the computer system 102 calculating the position of the third cardinal plane of the pelvis at step 230 using the position of the anatomical points and certain geometric constraints. In the described embodiment, the mid frontal part of the symphysis pubis lies 144 in both the pelvic frontal plane 146 and the mid sagittal pelvic plane 152. Hence, the mid frontal symphysis pubis point 144 and the two procesuss spinosus points 148, 152 between them define the mid sagittal pelvic plane 152. The mid frontal symphysis pubis point 144 and the spina iliaca anterior superior point 142 both lie in the pelvic frontal plane 146 and the pelvic frontal plane position can be determined using the geometrical constraint that the pelvic frontal plane and the mid sagittal pelvic plane and perpendicular. The transverse pelvic plane position can then be determined at step 230 from the further geometrical constraint that the transverse pelvic plane is perpendicular to both the pelvic frontal plane and the mid sagittal pelvic plane. Hence, in this approach certain geometrical constraints are used to augment the captured anatomical positions in order to allow the cardinal planes of the pelvis to be constructed or derived.

In the described embodiment four anatomical positions are used with one of the positions being common to two planes. However, in other embodiments more than four anatomical positions can be captured. Also, more than two anatomical positions in each plane can be captured. In general, three points are used to define a plane. In other embodiments, more than three positions can be used so as to try to more accurately or reliably define a plane. It will be appreciated that in embodiments in which a point common to two planes is not used, then, using three points to define the first plane, at least two more points and the constraint of being perpendicular to the first plane can be used to define the second plane. Hence, various combinations of numbers of points and geometric constraints can be used as will be apparent from the teaching herein.

After the position of the pelvis has registered with the CAS, by determining the positions of the cardinal planes, at step 232 further CAS processing operations can be carried out. For example, scan image data for use in IGS navigated procedures can be registered with the pelvis position data, by requiring the surgeon to identify positions in the previously captured image data of certain anatomical landmarks and then the CAS system registering the scan image data with the pelvis position, using the captured pelvis anatomical landmark position data. Hence, the scan image data can be presented to the surgeon on the display device 106 for use in IGS procedures and/or for used by subsequent planning and/or navigated procedures. Alternatively, a generic virtual pelvis model can be morphed and sized to fit to the captured anatomical point positions to provide a visual representation of the pelvis to aid the surgeon in IGS procedures and/or for use by subsequent planning and navigated surgical procedures. The registration method can then complete and return to the main surgical procedure at step 234.

With reference to FIG. 6, there is shown a flow chart illustrating a process 300 carried out by the computer system 102 to implement the pelvis registration method under control of a suitable computer program. The process is called and initiates at step 302. At step 304, the process determines the position of the marker attached to the patient at the iliac crest to track pelvic movement.

The computer program determines the identity of the marker attached to the instrument and from stored instrument data, determines the position of the instrument tip relative to the position of the marker. Using this information, the computer program can determine the position of the anatomical points corresponding to each of the captured instrument positions. At step 306, the program looks up the stored instrument position data corresponding to the spina iliac anterior superior point, computes the position of the anatomical point and saves data items representing this point in the co-ordinate frame of the tracking system. Then the program carries out the same operations at steps 308, 310 and 312 for the mid-frontal symphysis pubis, spinous process of S1 and spinous process of S2 respectively.

At step 314, the computer program calculates the position of the pelvic mid-saggital plane using the positions of the mid-frontal symphysis pubis, spinous process of S1 and spinous process of S2 points which all lie within this plane. Then at step 316, the program calculates the position of the pelvic frontal plane using the positions of the mid-frontal symphysis pubis and the spina iliac anterior superior, which both lie on a line in this plane, and together with the constraint that this plane is perpendicular to the pelvic mid-sagittal plane. Then at step 318, the computer program calculates the position of the transverse plane using the geometric constraint that this plane is perpendicular to both the pelvic frontal plane and the pelvic mid-sagittal plane. Hence, the program has determined the positions of the cardinal planes of the pelvis and so the physical position of the pelvis has been registered within the co-ordinate frame of the tracking system.

If the pelvis position is to be registered with pelvic image data or a virtual pelvis model then a process can be executed at step 320 to do so before process flow can return to a surgical workflow or other CAS application program at step 322. The cardinal planes can be used as references for calculating acetabular cup aneteversion and inclination angles.

It will be appreciated that the specific order in which the points are identified by the surgeon and captured by the tracking system is not vital in order for the invention to function. It will also be appreciated that the specific order in which the positions of points and/or the positions of planes are determined or calculated is also not vital.

FIG. 7 and the following discussion provide a brief, general description of an exemplary apparatus in which at least some aspects of the present invention may be implemented.

Various methods of the present invention have been described in the general context of computer-executable code or instructions, e.g., program modules, being executed by a computer device such as the computer system part 102 of the CAS system 100.

Some of the methods of the present invention may be effected by apparatus other than the specifically described computer devices. Software may include routines, programs, objects, components, data structures, etc. that perform a task(s) or implement particular abstract data types. Moreover, those skilled in the art will appreciate that at least some aspects of the present invention may be practised with other configurations, including hand-held devices, multiprocessor systems, microprocessor-based or programmable consumer electronics, network computers, minicomputers, mainframe computers, and the like. At least some aspects of the present invention may also be practised in distributed computing environments where tasks are performed by remote processing devices linked through a communications network. In a distributed computing environment, program modules may be located in local and/or remote memory storage devices.

With reference to FIG. 7, an exemplary apparatus 102 for implementing at least some aspects of the present invention includes a general purpose computing device, e.g., computer 400. The computer 400 may include a processing unit 402, a system memory 404, and a system bus 406 that couples various system components including the system memory 404 to the processing unit 402. The system bus 406 may be any of several types of bus structures including a memory bus or memory controller, a peripheral bus, and a local bus using any of a variety of bus architectures. The system memory 404 may include read only memory (ROM) 408 and/or random access memory (RAM) 410. A basic input/output system 412 (BIOS), including basic routines that help to transfer information between elements within the computer 400, such as during start-up, may be stored in ROM 408. The computer 400 may also include a hard disk drive 414 for reading from and writing to a hard disk, (not shown), a magnetic disk drive 416 for reading from or writing to a (e.g., removable) magnetic disk 418, and an optical disk drive 420 for reading from or writing to a removable (magneto) optical disk such as a compact disk or other (magneto) optical media. The hard disk drive 414, magnetic disk drive 416, and (magneto) optical disk drive 420 may be coupled with the system bus 406 by a hard disk drive interface, a magnetic disk drive interface, and a (magneto) optical drive interface, respectively. The drives and their associated storage media provide nonvolatile storage of machine readable instructions, data structures, program modules and other data for the computer 400. Although the exemplary environment described herein employs a hard disk, a removable magnetic disk 418 and a removable optical disk, those skilled in the art will appreciate that other types of storage media, such as magnetic cassettes, flash memory cards, digital video disks, Bernoulli cartridges, random access memories (RAMs), read only memories (ROM), and the like, may be used instead of, or in addition to, the storage devices introduced above.

A number of program modules may be stored on the hard disk, magnetic disk 417, (magneto) optical disk, ROM 408 or RAM 410, such as an operating system 422, one or more application programs 424, other program modules 426, and/or program data 428 for example. A user may enter commands and information into the computer 400 through input devices, such as a keyboard 430, touch screen 106 and pointing device 432 for example. Other input devices (not shown) such as a microphone, joystick, game pad, satellite dish, scanner, or the like may also be included. These and other input devices are often connected to the processing unit 402 through a serial port interface 434 coupled to the system bus 406. However, input devices may be connected by other interfaces, such as a parallel port, a game port or a universal serial bus (USB). A monitor 106 or other type of display device may also be connected to the system bus 406 via an interface, such as a video adapter 436 for example. In addition to the monitor 106, the computer 400 may include other peripheral output devices (not shown), such as speakers and printers for example.

The computer 400 may operate in a networked environment which defines logical connections to one or more remote computers. The remote computer may be another personal computer, a server, a router, a network PC, a peer device or other common network node, and may include many or all of the elements described above relative to the computer 400. The computer 400 can be connected to a remote computer by logical connections including a local area network (LAN), a wide area network (WAN), an intranet and the Internet.

When used in a LAN, the computer 400 may be connected to the LAN through a network interface adapter (or “NIC”) 438. When used in a WAN, such as the Internet, the computer 400 may include a modem 440 or other means for establishing communications over a wide area network. The modem 440, which may be internal or external, may be connected to the system bus 406 via the serial port interface 434. In a networked environment, at least some of the program modules, program data or data used by the programs depicted relative to the computer 400 may be stored in the remote memory storage device. The network connections described are exemplary and other means of establishing a communications link between the computers may be used.

It will be appreciated that the flowcharts illustrating the operations carried out are schematic and that certain operations may be omitted and/or the sequence changed or merged into other operations while still meeting the general teaching of the invention herein. Therefore the flowcharts are not intended to limit the invention only to the specific flow of processing and operations illustrated.

In view of the description of the invention included herein, numerous additional embodiments and variations on the discussed embodiments of the present invention will be apparent to one of ordinary skill in the art. It is to be understood that such embodiments do not depart from the present invention and are to be considered within the scope of the invention as defined in the appended claims.

1-44. (canceled)

45. A method for registering a pelvis of a subject in a lateral position, comprising:
determining the position of at least a first point of the pelvis and a second point of the pelvis in a first plane parallel to a first cardinal plane of the pelvis;
determining the position of at least a third point of the pelvis and a fourth point of the pelvis in a second plane parallel to a second cardinal plane of the pelvis, wherein the second plane is perpendicular to the first plane; and
determining the position of a third cardinal plane of the pelvis, wherein the third cardinal plane is perpendicular to the first cardinal plane and second cardinal plane.

46. The method of claim 45, wherein one of the first and second points or one of the third and fourth points is common to the first plane and the second plane.

47. The method of claim 45, wherein the first cardinal plane is the pelvic frontal plane.

48. The method of claim 45, wherein the second cardinal plane is the pelvic mid sagittal plane.

49. The method of claim 45, wherein the third cardinal plane is the transverse pelvic plane.

50. The method of claim 45, further comprising the step of determining the position of a fifth point of the pelvis in the first plane.

51. The method of claim 50, further comprising the step of determining the position of a sixth point of the pelvis in the second plane.

52. The method of claim 45, wherein the first plane is the first cardinal plane.

53. The method of claim 45, wherein the second plane is the second cardinal plane.

54. The method of claim 45, wherein determining the position of the first, second, third and fourth points includes tracking the position of an instrument bearing a marker detectable by a tracking system.

55. The method of claim 45, further comprising the step of registering the position of the pelvis with a virtual model of the pelvis or an image of the pelvis.

56. The method of claim 45, further comprising the step of attaching a marker detectable by a tracking system to determine the position of the marker to a specified anatomical feature of the pelvis.

57. The method of claim 45, wherein determining the position of the first, second, third and fourth points includes applying an end of an instrument to respective anatomical features of the pelvis.

58. The method of claim 57, wherein determining the position of the first, second, third and fourth points includes subcutaneously applying the end of the instrument.

59. The method of claim 57, wherein determining the position of the first, second, third and fourth points includes percutaneously applying the end of the instrument.

60. The method of claim 59, further comprising the step of palpating the skin of the subject to identify the anatomical features before applying the end of the instrument to the skin above the anatomical feature.

61. The method of claim 45, wherein determining the position of the third cardinal plane comprises calculating the position of the third cardinal plane.

62. The method of claim 45, wherein the first point is the spina iliaca anterior superior and the second point is the symphysis pubis.

63. The method of claim 45, wherein the third point is the spinous process of the S1 vertebra and the fourth point is the spinous process of the S2 vertebra.

64. The method of claim 46, wherein the point common to the first and second planes is the symphysis pubis.

65. A system for registering the pelvis of a patient in a lateral position, comprising:
an instrument for locating a plurality of anatomical points of, or adjacent, the pelvis and wherein the position of the instrument is detectable by a tracking system;
a tracking system operable to generate an instrument position signal indicative of the position of the instrument; and
a computer system in communication with the tracking system to receive the instrument position signal, and wherein the computer system includes a data processing device in communication with a memory, the memory storing instructions causing the data processing device to:
determine the position of a first pelvic plane from a first set of pelvic part positions derived from corresponding instrument positions;
determine the position of a second pelvic plane from a second set of pelvic part positions derived from corresponding instrument positions; and
determine the position of a third pelvic plane, wherein the first, second and third pelvic planes are mutually perpendicular.

66. The system of claim 65, wherein the instrument has a marker detectable by the tracking system.

67. The system of claim 66, wherein the marker is wirelessly detectable by the tracking system.

68. The system of claim 65, wherein the instrument position signal comprises data items representative of the position of the instrument.

69. The system of claim 65, wherein the tracking system and computer system are integrated.

70. The system of claim 65, wherein the first set of pelvic positions includes at least three pelvic positions in a first plane parallel to the first pelvic plane.

71. The system of claim 70, wherein the second set of pelvic positions includes at least two pelvic positions in a second plane parallel to the second pelvic plane.

72. The system of claim 71, wherein a one of the pelvic positions in the first set of pelvic positions and the second set of pelvic positions is common.

73. The system of claim 65, wherein the first set of pelvic part positions are in the first pelvic plane.

74. The system of claim 65, wherein the second set of pelvic part positions are in the second pelvic plane.

75. The system of claim 65, wherein the first set of pelvic part positions includes the positions of the spina iliaca anterior superior and the symphysis pubis.

76. The system of claim 65, wherein the second set of pelvic part positions includes the positions of the spinous process of the S1 vertebra and the spinous process of the S2 vertebra.

77. A method for registering a pelvis of a subject in a lateral position, comprising:
calculating the position of a first cardinal plane of the pelvis using the position of at least a first point and a second point located in a first plane parallel to the first cardinal plane;
calculating the position of a second cardinal plane of the pelvis using the position of at least a third point and a fourth point located in a second plane parallel to the second cardinal plane; and
calculating the position of a third cardinal plane of the pelvis, wherein the first, second and third cardinal planes are mutually perpendicular.

78. The method of claim 77, wherein the first plane is coincidental with the first cardinal plane and/or the second plane is coincidental with the second cardinal plane.

79. The method of claim 77, wherein the first cardinal plane is the frontal pelvic plane and/or the second plane is the sagittal pelvic plane and/or the third plane is the transverse pelvic plane.

80. The method of any of claims 77, wherein calculating the position of the second cardinal plane also uses a one of the first or second points.

81. The method of claim 80, wherein the first or second point is the symphysis pubis.

82. A method for registering the pelvis of a subject in a lateral position, the method comprising:
detecting the position of an instrument at a first, second, third and fourth position;
determining the position of a first, second, third and fourth anatomical feature of, or adjacent, the pelvis based on the respective detected positions; and
calculating the position of first, second and third mutually orthogonal cardinal planes of the pelvis.

83. The method of claim 82, wherein the detecting the position of the instrument includes wirelessly tracking the instrument.

84. The method of claim 83, wherein three of the first, second, third and fourth anatomical features lie in the same plane.

85. The method of any of claim 84, wherein calculating the position further comprises using a one of the first, second, third and fourth anatomical features which does not lie in the same plane and the geometric constraint that a two of the cardinal planes are perpendicular.
